(19) United States
(12) Patent Application Publication
(10) Pub. No.: US 2022/0354373 A1
(43) Pub. Date: Nov. 10, 2022

(54) ENERGY HARVESTING FOR WIRELESS SUBJECT MONITORING SENSOR

(71) Applicant: Hill-Rom Services, Inc, Batesville, IN (US)

(72) Inventors: Gavin M. Monson, Oxford, OH (US); Douglas A. Seim, Okena, OH (US)

(21) Appl. No.: 17/738,162

(22) Filed: May 6, 2022

Related U.S. Application Data
(60) Provisional application No. 63/186,361, filed on May 10, 2021.

(51) Int. Cl. A61B 5/0205; A61B 5/145; A61B 5/11; A61B 5/318; A61B 5/369; H02J 50/00

(52) U.S. Cl. CPC A61B 5/02055; A61B 5/14546; A61B 5/14542; A61B 5/11; A61B 5/318; A61B 5/369; H02J 50/001; A61B 2560/0209; A61B 2560/0214

(57) ABSTRACT

A sensor apparatus configured to detect at least one physiological parameter of a subject includes a sensor that detects at least one physiological parameter of the subject as sensor data. An energy harvesting circuit includes a plurality of energy harvesting devices configured to harvest ambient energy from an environment of the subject. The energy harvesting devices generate power at a plurality of voltage potential levels from ambient energy. A conditioning circuit is configured to adjust the plurality of voltage potential levels to a bus voltage supplied to a supply bus. A controller receives operating power via the supply bus and controls the activation of the sensor and the wireless communication circuit.

### CROSS-REFERENCE TO RELATED APPLICATIONS

This application claims priority under 35 U.S.C. § 119(e) and the benefit of U.S. Provisional Application No. 63/186,361, entitled ENERGY HARVESTING FOR WIRELESS SUBJECT MONITORING SENSOR, filed on May 10, 2021, by Gavin M. Monson, et al., the entire disclosure of which is incorporated herein by reference.

### FIELD OF THE DISCLOSURE

The present disclosure generally relates to a monitoring device for a subject and, more particularly, to a wireless monitoring apparatus comprising an energy harvesting device for prolonged operation.

### SUMMARY OF THE DISCLOSURE

According to one aspect of the present disclosure, a sensor apparatus configured to detect at least one physiological parameter of a subject is disclosed. The apparatus includes at least one sensor configured to detect the at least one physiological parameter of the subject as sensor data. A wireless communication circuit is configured to wirelessly communicate the sensor data. The sensor apparatus further includes an energy harvesting circuit with a plurality of energy harvesting devices configured to harvest ambient energy. The energy harvesting devices generate power at a plurality of voltage potential levels from the ambient energy. At least one conditioning circuit is configured to adjust the plurality of voltage potential levels to a bus voltage supplied to a supply bus. At least one controller receives operating power via the supply bus and controls an activation of the at least one sensor and the wireless communication of the sensor data.

According to another aspect of the disclosure, a system for a sensor apparatus configured to detect at least one physiological parameter of a subject is disclosed. The system includes at least one sensor disposed in the sensor apparatus configured to detect the at least one physiological parameter of the subject as sensor data. A wireless communication circuit is disposed in the sensor apparatus and configured to wirelessly communicate the sensor data to a computerized device. An energy harvesting circuit is also disposed in the sensor apparatus and configured to exclusively supply power to the sensor apparatus. The harvesting circuit includes a plurality of energy harvesting devices that harvest energy from at least two different forms of ambient kinetic energy. At least one controller of the system is configured to monitor the energy harvested by the plurality of harvesting devices over a periodic time interval. The at least one controller further predicts a power harvesting rate for the energy harvesting devices over the periodic interval and controls the at least one sensor and the wireless communication circuit with operating power less than the power harvesting rate.

According to another aspect of the disclosure, a method for operating a sensor apparatus configured to detect at least one physiological parameter of a subject is disclosed. The method includes monitoring the at least one physiological parameter of the subject as sensor data at a detection frequency with the sensor apparatus and wirelessly communicating the sensor data at a communication frequency. The method further comprises harvesting energy with a plurality of energy harvesting devices. The energy harvesting devices generate power at a plurality of voltage potential levels from ambient energy. The energy harvested by the energy harvesting devices is the exclusive power supplied to the sensor apparatus. The method further includes adjusting the plurality of voltage potential levels to a bus voltage supplied to a supply bus and supplying the bus voltage to a controller of the sensor apparatus. The controller also controls the detection frequency and the communication frequency.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is an exemplary diagram demonstrating a clinical environment implementing a patient monitoring device;

FIG. 2 is a schematic diagram demonstrating a patient monitoring device and corresponding monitoring system in communication with a mobile device and remote server;

FIG. 3 is a block diagram of a patient monitoring device including a plurality of energy harvesting devices;

FIG. 4 is a schematic diagram of a cumulative energy harvesting circuit configured to capture ambient energy via a plurality of energy harvesting devices;

FIG. 5A is a plot demonstrating an average power harvested by a plurality of energy harvesting devices;

FIG. 5B is a plot demonstrating a power management plot demonstrating an average power utilized to operate the patient monitoring device;

FIG. 6A is a flow chart demonstrating a power management routine for operating a patient monitoring device; and

FIG. 6B is a flow chart demonstrating a power management routine for operating a patient monitoring device continued from FIG. 6A.

### DETAILED DESCRIPTION

The present illustrated embodiments reside primarily in combinations of method steps and apparatus components related to a patient monitoring device. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

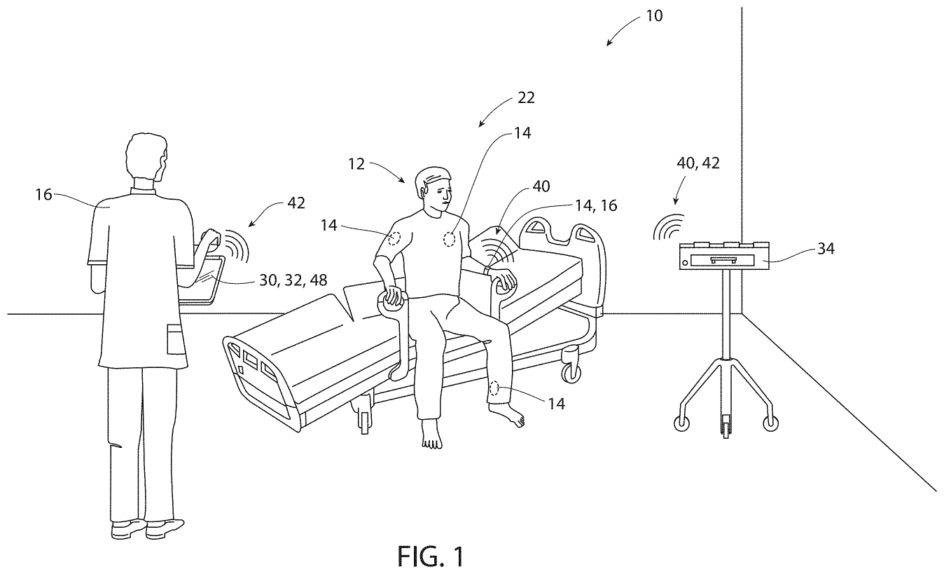

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

Referring to FIGS. 1-3, reference numeral 10 generally designates a patient monitoring system. The monitoring system 10 provides for the collection and analysis of physiological data of patients 12 via a monitoring device 14. The monitoring system 10 provides various benefits to patients and clinicians. In particular, the monitoring system 10 provides for continuous monitoring without restricting the movement of the patient 12. In contrast, conventional monitoring devices may require invasive, wired connections to large stationary electronic devices. Such devices restrict the movement of patients 12 and are commonly associated with discomfort. The monitoring device 14 provides for wireless operation that may be sustained for extended periods without requiring a wired connection for charging or data communication.

As illustrated in FIG. 1, the monitoring device 14 is depicted as a wearable device 16 having a wrist band connecting the device 14 to the patient 12. Additionally, the wearable device 16 may be adhered to, implanted, or otherwise connected to the patient 12. The monitoring device 14 may also be implemented as multiple monitoring devices 14 in connection with various portions or parts of the patient 12. For example, the monitoring system 10 may correspond to a distributed system or network of devices that are connected to the patient and record physiological data in concert. Accordingly, the system 10 may be flexibly implemented to communicate various biological, vital, or physiological information from the patient 12 for review by clinicians 16, physicians, and medical staff to monitor the health and behavior of the patient 12.

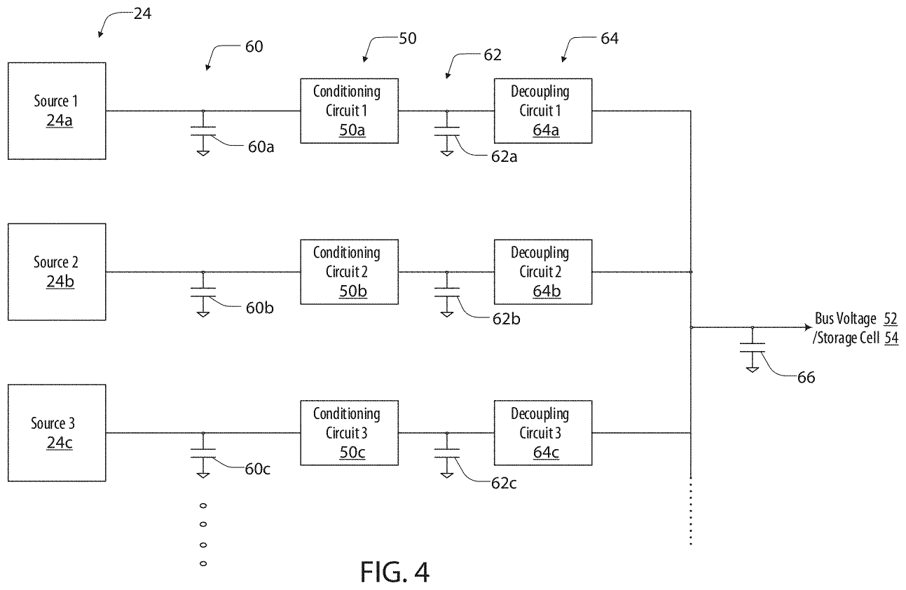

To sustain the operation of the monitoring device 14, the disclosure provides for an energy harvesting circuit 20 that operates based on ambient energy harvested from a local environment 22 proximate to the patient 12. In order to maximize the energy harvested from the local environment, the energy harvesting circuit 20 captures energy from a plurality of energy harvesting devices 24. Each of the energy harvesting devices 24 act independently to capture and convert ambient kinetic or environmental energy sources into usable energy for the monitoring device 14. For example, the harvesting devices 24 may include a combination of two or more energy sources that derive energy from photovoltaic sources, magnetic induction sources, piezoelectric sources, thermoelectric sources, radio frequency sources, electromagnetic energy sources, or other ambient energy sources. Accordingly, the system 10 is configured to sustain the operation of a plurality of sensors 26 and a communication circuit 28 for extended periods. In this way, the monitoring device provides for the detection and communication of the sensor data recorded by the sensors 26 for extended periods. While the period for self-sustained operation may vary, the system 10 is designed to operate without the need for manual charging or replacement for the duration of a stay or term of treatment of the patient 12. Accordingly, the monitoring device 14 is configured to operate self-sufficiently for months or longer. The nature of the combined operation of the harvesting devices 24 and the operation of the harvesting circuit 20 are further discussed in reference to FIG. 4.

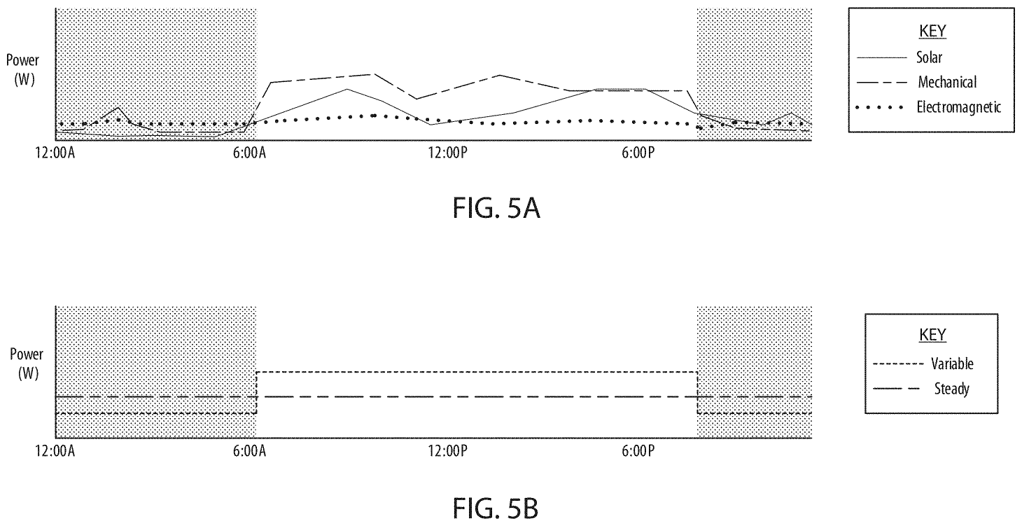

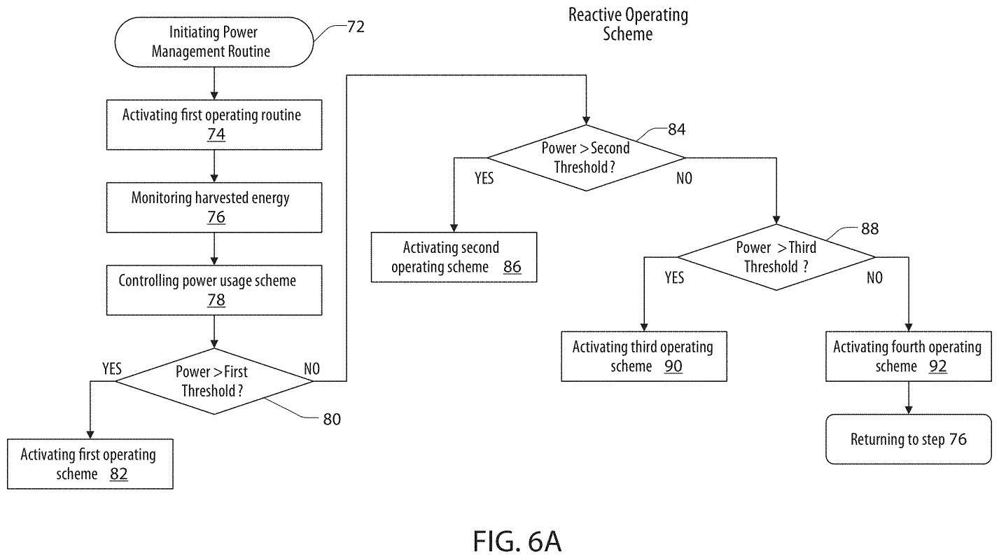

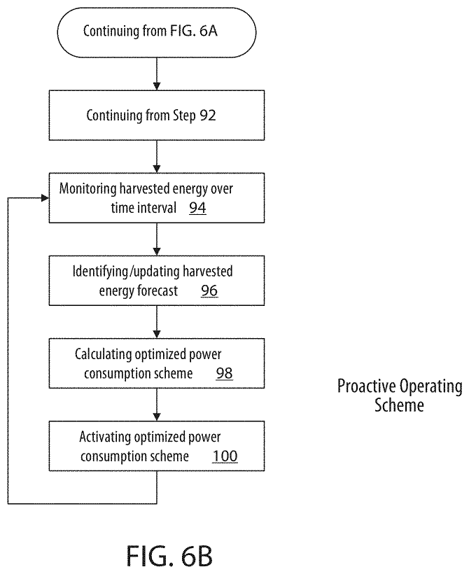

The sensors 26 may correspond to physiological or biological monitoring sensors of the monitoring device 14 and may vary based on the specific application or conditions of the patient 12 to be monitored. As a result of the variations of the sensors 26 and the communication needs for different patients, the operational power consumption of the monitoring device 14 may also vary. When these variables are coupled with the natural variations associated with the harvesting of ambient or waste energy, sustaining the operation of the monitoring device 14 may involve a balance of monitoring the harvested power while managing the operation of the monitoring device 14 to operate with only the harvested power. The disclosure provides for novel operating methods for the harvesting circuit 20 to maintain the operation of the monitoring device 14. In some implementations, a controller 30 of the monitoring device utilizes a learning operation to adjust the operation the sensors 26 and the communication circuit 28 in order to sustain operation based on the availability of ambient or waste energy. Additionally, complementary harvesting devices 24 are applied to optimize harvesting for various operating environments. Accordingly, the operation of the monitoring device 14 and the sustained operation of the system 10 are supported by various operating routines and configurations of the monitoring device 14 provided by the disclosure. Further details of power management routines are discussed in reference to FIGS. 5A, 5B, 6A, and 6B.

The sensors 26 may incorporate various forms of instrumentation, which may be incorporated in a single integrated sensor module (e.g., the wearable device 16) and/or a plurality of distributed devices each in communication with the system 10 via one or more wireless communication protocols. The sensors 26 may include, but are not limited to, electrocardiographs (EKG), electroencephalographs (EEG), acoustic sensors, tissue and blood oxygen level sensors, blood chemistry sensors, surface or body temperature sensors, accelerometry sensors (e.g., patient movement or determination of patient orientation), etc. In implementations where the sensors 26 are implemented as separate modules, the system 10 may provide for modular integration of the monitoring devices 14 as disparate modular units that communicate information from the various sensors implemented.

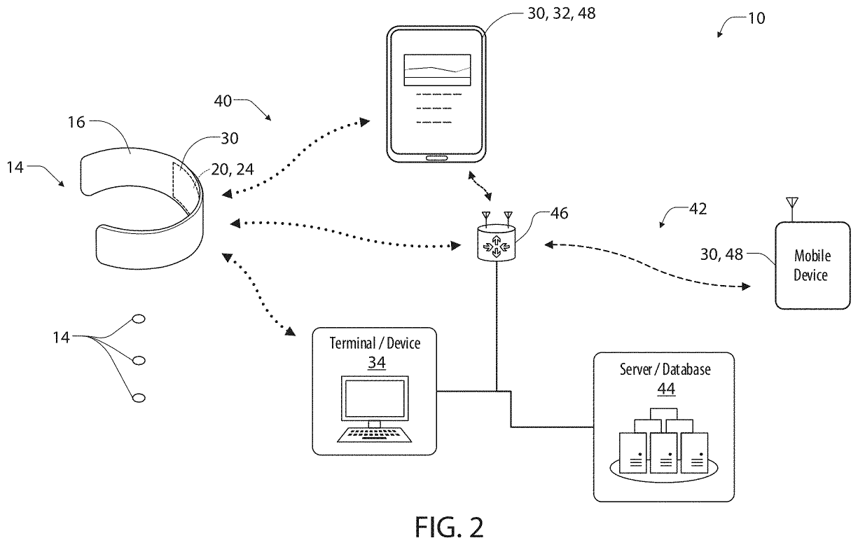

To support the wireless communication of the monitoring device 14, the system 10 may communicate with the monitoring device 14 via one or more intermediate communication devices or network devices, which may be in the form of mobile devices 32, tablets 34, computer terminals 36, etc. Each of the intermediate communication devices may be in communication via a first wireless communication interface 40. As demonstrated in FIG. 2, the first wireless communication interface 40 may be one of a plurality of communication interfaces, which may be implemented via a variety of communication protocols. The first wireless communication interface 40 may correspond to a local communication interface, which may communicate with a second communication interface 42 or a hospital network. The second communication interface 42 may include a combination of wired connections (e.g., Ethernet) as well as wireless network interfaces.

In order to support limited energy consumption, the first communication interface 40 may correspond to a local or short-range communication protocol that communicates with the second communication interface 42 via one or the intermediate devices (e.g. the mobile devices 32, tablets 34, computer terminals 36, etc.). In such implementations, the first communication interface 40 may utilize Bluetooth®, Bluetooth® Low Energy (BLE), Thread, Ultra-Wideband, Z-Wave, ZigBee, or similar communication protocols. The second communication interface 42 may correspond to different wireless communication protocol that the first communication interface including, but not limited to, global system for mobile communication (GSM), general packet radio services (GPRS), code division multiple access (CDMA), enhanced data GSM environment (EDGE), fourth-generation (4G) wireless, fifth-generation (5G) wireless, Wi-Fi, world interoperability for microwave access (WiMAX), local area network (LAN), Ethernet, etc. Though discussed as implementing different wireless communication protocols, the first and second communication interfaces 40, 42 may alternatively be implemented as a single, common interface and protocol. By flexibly implementing the wireless communication interfaces 40, 42, the monitoring system 10 can be in communication with one or more of the remote mobile devices 32 and the remote server 44 directly, via a router 46 and/or via a cellular data connection.

In operation, the sensor data is recorded by and communicated from the monitoring device 14 to the first communication interface 40 or the second communication interface 42. In response to the communication from the monitoring device 14, the intermediate devices (e.g., the mobile devices 32, tablets 34, computer terminals 36, etc.) or other computerized systems may process the sensor data and notify the staff or medical professionals of conditions or vital information recorded from the patient 12. As shown in FIG. 2, the information captured by the monitoring device 14 may be communicated to the server or remote database 44 via the second communication interface 42, which may be communicated via the wireless router 46 network switch or various other network communication utilities. The sensor data and the resulting notifications, alerts, or communicated information regarding the patient 12 are further communicated to additional portable electronic devices 48 in communication with the second wireless interface 42 of the system 10.

As provided by the disclosure, the monitoring device 14 may be implemented as a passive reporting device that communicates the data recorded by the sensors 26. The term passive is used in relation to reporting the sensor data in contrast with a response provided by the system 10 that may result from processing and reviewing the sensor data. For example, the aspects related to processing, tracking, and/or activating alerts or alarms may be processed by one of more of the intermediate devices (e.g., the mobile device 32, tablet 34, computer terminal 36, etc.) or other devices in communication with the first or second communication interfaces 40, 42. By limiting data processing and computing tasks, the monitoring device 14 may improve operational efficiency by periodically deactivating the operation of one or more of the sensors 26 and/or limiting the communication frequency from the monitoring device 14. As later discussed, the data communication frequency and sampling or read frequency of the output signals from the sensors 26 may be controlled and prioritized to limit power consumption. Such prioritization and control of the sensors 26 allow the system 10 to operate effectively despite irregularities or shortages that may be associated with the energy harvested via the harvesting devices 24.

In addition to responsively adjusting the operating capabilities and performance of the monitoring device 14 to preserve operation, the system 10 may also provide for the controller 30 of the device 14 to proactively adjust the operation of the sensors 26 and the communication circuit 28. For example, the controller 30 may adjust the operation of the sensors 26 and the communication circuit 28 based on a forecast or predicted energy harvesting schedule determined for the specific patient 12. As later discussed in reference to FIGS. 5A, 5B, 6A, and 6B; the controller 30 of the monitoring device 14 may modulate the power consumption of the sensors 26 and the communication circuit 28 by adjusting the frequency of capturing and/or reporting the sensor data to the system 10. Additionally, the controller 30 may prioritize and selectively control the activation of each of the sensors 26 based on the energy made available from the harvesting devices 24 and resulting from the behavior and the local environment of the patient 12.

In some instances, the system 10 may monitor the activity, behavior, and schedule of the patient 12 as well as the harvesting performance of each of the harvesting devices 24 in response to the environment and behavior of the patient 12. Based on the information identified from the monitoring device 14, the system 10 (e.g., the controller 30, the mobile device 32, tablet 34, computer terminal 36, etc.) may identify a forecast for the average energy harvested by the harvesting devices 24. Based on the forecast of the harvested energy captured by the harvesting devices 24, the system 10 may adjust the operation of the sensors 26 and the communication circuit 28 of the system 10 to maximize the operation of the monitoring device 14 based on the available power from the harvesting devices 24. Additionally, the system 10 may provide for a variety of control schemes for the monitoring device 14 in response to the availability or shortages of the harvested energy.

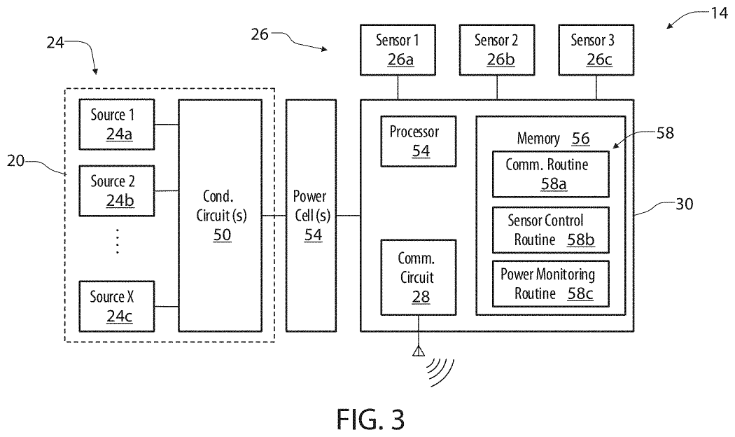

Referring now to FIGS. 3 and 4, the monitoring device 14 and the harvesting circuit 20 are discussed in further detail. As previously discussed, the type and quantity of the sensors 26 and the harvesting devices 24 may vary based on the application. As depicted in FIGS. 3 and 4, the monitoring device 14 includes 2 or more harvesting devices 24 that derive energy from different ambient or waste energy sources. To clearly refer to each of the harvesting devices 24, the devices are referred to as a first energy source 24a, a second energy source 24b, a third energy source 24c, etc. In operation, the energy harvested from each of the harvesting devices 24 may be converted via one or more conditioning circuits 50, which may convert the power derived from each of the harvesting devices 24 to a common or bus voltage 52. In this configuration, the bus voltage 52 is conducted to a storage cell 54 from which the controller 30 of the monitoring device 14 may access operating power and supplies energy to control the operation as discussed herein.

The controller 30 may include various components and/or integrated circuits to provide for the control of the monitoring device 14. The controller 30 may include various types of control circuitry, digital and/or analog, and, as shown, includes a processor 55, which may be implemented as a microcontroller, application-specific integrated circuit (ASIC), or other circuitry configured to perform various input/output, control, analysis, and other functions as described herein. The controller 30 further includes a memory 56 configured to store one or more control routines 58, including a communication control routine 58a, a sensor control routine 58b, and/or a power monitoring routine 58c. Each of the control routines 58 includes operating instructions to enable the methods discussed herein and may be updated by communication with various components of the system 10 (controller 30, the mobile device 32, tablet 34, computer terminal 36). The memory 56 can be implemented by a variety of volatile and non-volatile memory formats. Additionally, the communication circuit 28 may be configured to communicate via various wireless communication protocols as discussed in reference to each of the first communication interface 40 and the second communication interface 42. Accordingly, the controller 30 permits communication to and from the monitoring device 14 via a variety of communication protocols including various protocols that are yet to be discovered at the time of this disclosure.

The sensors 26 may correspond to various types of sensors configured to detect physiological or biological information of the patient 12 and report the information to the controller 30 for communication via the communication circuit 28 to the system 10. The physiological information may correspond to data internal to the patient, such as vital data or other information that may not be readily associated with voluntary patient activity. Additionally, in some cases, information identifying the motion or interaction of the patient 12 with the local environment may be recorded by the sensors 26. The sensors 26 may include, but are not limited to, electrocardiogram (EKG), electroencephalogram (EEG), acoustics, tissue and blood oxygen levels, blood chemistry, surface or body temperature, and accelerometry (e.g., patient movement, impact, or determination of patient orientation), etc. In implementations where the sensors 26 are implemented as separate modules, the system 10 may provide for modular integration of the monitoring devices 14 as disparate modular units that communicate information from the various sensors implemented to the system 10 as discussed herein.

As previously discussed, the harvesting devices 24 may be configured to harvest various forms of ambient kinetic energy (e.g., radiant energy, thermal energy, motion energy, etc.). Some examples of the harvesting devices 24 include photovoltaic sources, magnetic induction sources, piezoelectric sources, thermoelectric sources, radio frequency sources, electromagnetic energy sources, etc. More specifically, the harvesting devices 24 may include, but are not limited to, mechanical (piezoelectric, including flexible polymer piezoelectric materials, PZT materials and others); thermoelectric (Seebeck effect via a Peltier effect device); solar/light power conversion from ambient light and sunlight; and RF capture of ambient RF fields, most likely in a parasitic mode, which is to say energy from other Bluetooth® devices, Wi-Fi beacons nearby, and potentially RFID signals from devices such as wireless incontinence detection are potential sources of energy. Other non-parasitic energy sources for the harvesting devices 24 include wireless power transfer signals (e.g. capacitive, inductive) embedded in the surface of a patient support device, or potentially an RFID system included for the purpose of providing energy for the monitoring device 14. Accordingly, the system 10 is configured to sustain the operation of the sensors 26 and the communication circuit 28 for extended periods with only the power harvested by the harvesting devices 24.

The nature of the diverse utilization of disparate energy sources for the harvesting devices 24 may result in power supply variations as well as inconsistencies in voltage levels supplied by the harvesting devices 24. For example, the level of voltage from some of the harvesting devices 24 may be very low and inadequate or deficient for direct use by the controller 30. Additionally, the output from other harvesting devices 24 of the monitoring device 14 may present excessively high voltage relative to the desired supply voltage. In some cases, the current supplied by the harvesting devices 24 may also differ from the supply voltage desired. Accordingly, the harvesting circuit 20 provides for storage and conversion of the energy from the diverse harvesting devices 24, such that the cumulative energy can be combined into a single energy reservoir suitable for use by monitoring device 14. FIG. 4 demonstrates an exemplary block diagram of the harvesting circuit 20 configured to combine the energy supplied by each of the harvesting devices 24.

Referring to FIG. 4, the harvesting circuit 20 includes a raw storage unit 60 (e.g., capacitor) in connection with each of the harvesting devices 24, denoted as the first energy source 24a, the second energy source 24b, a third energy source 24c, etc. Each of the raw storage units 60 is configured to store raw electrical energy harvested by the corresponding harvesting device 24. For clarity, a first raw storage unit 60a is in connection with the first energy source 24a, a second raw storage unit 60b is in connection with the second energy source 24b, and a third raw storage unit 60c is in connection with the third energy source 24c. The terms first, second, third, etc. as discussed herein are for clarity in reference to the illustrated examples and should not be considered limiting to a specific quantity or priority of the recited elements. Accordingly, though three harvesting devices 24 are described in the exemplary implementations, the invention is not so limited.

Each raw storage unit 60 is further connected to at least one voltage conditioning circuit 50, as previously discussed. In various implementations, the voltage conditioning circuits 50 are in connection with each of the raw storage units 60 as dedicated voltage conversion circuits that convert the voltage from the corresponding energy harvesting device 24 to the bus voltage 52. More specifically, the first raw storage unit 60a is in connection with a first conditioning circuit 50a, the second raw storage unit 60b is in connection with a second conditioning circuit 50b, the third raw storage unit 60c is in connection with a third conditioning circuit 50c, etc. Each of the conditioning circuits may include a voltage conversion circuit, which may be implemented as magnetic converters, resonant converters, switching converters, or various forms of voltage conversion circuits. In some cases magnetic DC-to-DC converters are implemented in the form of step-down (buck) converters, step-up (boost) converters, single-ended primary-inductor converters (SEPIC), or other voltage converters/signal processing circuits. In this configuration, voltages that are higher or lower than the bus voltage 52 collected in each of the raw storage units 60 are converted to the bus voltage 52.

Each of the conditioning circuits 50 is shown in connection with a converted voltage storage unit 62 (e.g., capacitor) that supplies the converted voltage from the conditioning circuits 50 to a decoupling circuit 64. Each decoupling circuit 64 is connected to a common bulk storage unit 66 (e.g., capacitor) configured to supply the bus voltage 52. For clarity, a first converted voltage storage unit 62a couples the first conditioning circuit 50a to a first decoupling circuit 64a, a second converted voltage storage unit 62b couples the second conditioning circuit 50b to a second decoupling circuit 64b, and a third converted voltage storage unit 62c couples the third conditioning circuit 50c to a third decoupling circuit 64c. Each decoupling circuit 64a, 64b, 64c, etc. supplies the converted and filtered voltage to the bulk storage 66 to supply the monitoring device 14 with operating power.

The decoupling circuits 64 are configured to prevent fluctuations in voltage supplied from each of the disparate conditioning circuits 50 to the power bus voltage 52. The decoupling circuits 64 may be implemented as low voltage drop pass circuits consisting of directional switches (e.g., MOSFETs) with corresponding control circuits. Such devices control the conduction of energy and limit losses resulting from energy dissipation to promote efficient use of the harvested energy. Essentially, the decoupling circuits 64 operate as actively controlled diodes with an extremely low forward voltage drop from the conditioning circuits 50 to the bulk storage unit 66. The bulk storage unit 66 serves as the input power to the monitoring device 14 and may include a further voltage regulation circuit. In operation, the energy captured by each harvesting device 24 is stored and converted to a common voltage used for the bus voltage 52 of the monitoring device 14. The decoupling circuits 64 optimize the transfer of energy from the voltage conditioning circuits 50 to the supply of the bus voltage 52 by ensuring that energy supplied to the bulk storage unit 66 is not coupled back to the output of the voltage conditioning circuits 50.

Conceptually, the harvesting circuit 20 may be configured to operate as a charge pump device. That is, the harvesting circuit is configured to receive the output of the energy harvesting devices 24 at their native output voltage and current levels and convert the harvested voltages to the bus voltage 52. The harvesting circuit 20 may typically be implemented in a pulse mode that converts energy as it becomes available from the harvesting devices 24 and temporarily stores the energy in the raw storage units 60. However, if energy adequate for conversion is consistently supplied from one or more of the harvesting devices 24, the conversion and supply to the bus voltage 52 may be continuously converted and supplied. In some cases, fluctuations in energy availability from the harvesting devices 24 may vary, such that the energy supplied by the harvesting circuit 20 varies significantly over time. In such cases, surplus energy may be stored in the storage cell(s) 54 (e.g., capacitor, rechargeable cell, solid-state battery, etc.) to ensure that energy is supplied to the monitoring device 14 during droughts or shortages in energy harvested or scavenged by the harvesting devices 24.

Referring now to FIGS. 5A and 5B, plots are shown demonstrating an average power harvested by a plurality of the energy harvesting devices 24 and an average power utilized to operate the monitoring device 24, respectively. It is understood that variations in the energy supplied from the harvesting circuit 20 and the variations in usage by the monitoring device 14 may vary more significantly than that shown in FIGS. 5A and 5B. However, the plots are representative of the average power harvested and utilized as detected by the system 10 over time. The average power conditions of the system are more meaningful when considered in reference to the harvesting and discharge of power from the bulk storage unit 66 or battery in connection with the bus voltage 52. As demonstrated in FIG. 5A, the power harvested from the harvesting devices 24 is recovered from two or more different energy sources. That is, the energy is not only harvested from separate devices but from different ambient sources. In the example shown, the harvesting devices 24 include a photoelectric cell configured to harvest solar energy, a piezoelectric transducer configured to harvest mechanical energy, and an electromagnetic transducer configured to harvest ambient radiation. Though the harvesting device 24 is discussed in reference to these energy sources, the disclosure is not limited to any specific quantity or combination of devices.

As shown in FIG. 5A, the average power harvested from each of the harvesting devices 24 varies due to the environment surrounding the patient 12 as well as the movement or activity of the patient 12. Accordingly, the power available from the harvesting devices 24 will differ for each monitoring device 14 and patient 12, which causes a significant increase in operational complexity when compared to conventional electronic devices. As shown, the electromagnetic energy harvested may be the most consistent as a result of limited variation in the electromagnetic activity in the local environment. In contrast, the energy harvested from the mechanical and solar sources may vary more significantly based on the ambient lighting conditions as well as the movement of the patient 12. Accordingly, the available energy may vary significantly based on the types of energy recovered by the harvesting devices 24, the local environment of the patient 12, and the activity of the patient 12. These variations are not only energy source dependent but also patient dependent and may drastically vary over time. Due to these variations, the harvesting capacity of the harvesting circuit 20 may vary widely such that ensuring a steady supply of energy to the monitoring device 14 may not be possible.

To accommodate for the variations in power harvested by the harvesting circuit 20, the disclosure provides for the controller 30 to vary the operation of the monitoring device 14 based on the available or predicted/forecast energy or power available from the harvesting circuit 20. For example, if the average power supplied by the harvesting circuit 20 is identified as the sum of the harvesting devices 24 as shown in FIG. 5A, the corresponding power available from the harvesting circuit 20 is shown in FIG. 5B. As shown in the “variable” power line, the power available increases during periods of physical activity of the patient 12 and during bright ambient conditions, which correspond to the hours between 6:00 AM and 8:00 PM. As discussed in reference to FIGS. 6A and 6B, the system 10 may detect and/or anticipate such fluctuations in the rate of power harvested by the harvesting devices 24 and responsively adjust the operation of the monitoring device 14.

In some cases, the system 10 may monitor the power harvested by the harvesting circuit 20 and calculate or forecast an average power or “steady” power expected to be available from historical power data observed for the harvesting device 24. Based on the “steady” power calculated as plotted in FIG. 5B, the system 10 may adjust operation to ensure that adequate energy is available to sustain the operation of the monitoring device. For example, the operation may be adjusted by the controller 30 or system 10 by controlling one or more of the communication frequencies of the communication circuit 28 and the timing/frequency of reading or activating the sensors 26 to limit the power consumption of the monitoring device 14. In some cases, the operation of one or more of the sensors 26 may be temporarily or permanently suppressed to prioritize the reporting of one or more of the sensors 26. Put differently, the reading and reporting frequencies of the sensor data from the patient 12 may be varied by the controller 30 for each of the sensors 26 to ensure that the power supplied by the harvesting circuit 20 is sufficient to operate the monitoring device 14. Methods related to the adjustment of operation of the monitoring device are discussed in further detail in reference to FIGS. 6A and 6B.

Referring now to FIGS. 6A and 6B, a method 70 for controlling the operation of the monitoring device 14 is shown. For clarity, the method 70 includes various steps that could be further clarified or, in some cases, omitted. For example, the operating schemes may be increased or limited depending on the complexity of the monitoring device 14, the number of sensors 26, etc. Also, it shall be understood that the exemplary control characteristics (e.g., communication frequency, operating frequency, deactivation of sensors, etc.) of the operating schemes may be rearranged in priority according to the relative importance of the information being reported by each of the sensors 26 for each application. For example, the operation of certain sensors may be more important when monitoring patients with specific conditions. Accordingly, the operating schemes may be prioritized and set for operation based on the treatment and monitoring needs for each patient. Accordingly, the method 70 provides for options that can be implemented to adjust the operation of the system 10 based on the priority of the information reported and the frequency of the information required for specific patients or groups of patients with similar conditions.

In some cases, the system 10 may monitor sensor data from one or more of the sensors 26 and compare the data to various vital statistics or health markers for the associated patient. The health markers may correspond to predetermined levels (e.g., blood-oxygen level, heart rate, body temperature, etc.) of the sensor data reported for each patient. In response to changes in the sensor data, the system 10 may control the sensors 26 to adjust the operating characteristics of the monitoring device 14. For example, in response to detecting the sensor data reporting patient information exceeding or below one of the health markers (e.g., greater than or less than a predetermined or preset threshold), the system 10 may adjust the operating scheme of the monitoring device 14 based on a patient care priority. In doing so, the system 10 may activate or deactivate one or more of the sensors 26 and adjust the communication or reporting frequency of the communication circuit 28. Accordingly, the system 10 may detect changes in the sensor data reported by the sensors 26 and adjust the operation (e.g., sensor activation, communication timing, etc.) in order to ensure that the critical information for the patient is effectively reported. In other words, the system 10 may adjust an operating routine of the monitoring device 14 in response to changing conditions of the patient detected in the sensor data.

In some cases, the priority of operation of the sensors 26 identified based on the sensor data may even cause the system 10 to activate operation that cannot be sustained by the harvesting circuit 20. In such cases, the system 10 may prioritize reporting extensive sensor data (e.g. increased frequency and sensor activation) even if the operation will result in an eventual power shortage due to the discharge of the stored energy. Such operation may be activated in order to ensure that important sensor data is reported for a period before the operation of the monitoring device 14 can no longer be sustained by the harvesting circuit 20 and the device 14 shuts-down. This operation may be referred to as a data reporting prioritized configuration that can be activated to report important sensor data for an interim period before alternative, conventional monitoring devices (e.g., AC powered) can be connected to the patient. Additionally, the activation of this type of operation of the monitoring device 14 may be accompanied by an alert indicating that alternative sensory equipment may be necessary to provide care for the patient.

The method 70 may begin in step 72 in response to the activation of the monitoring device 14. Upon activation, the controller 30 of the monitoring device 14 may initiate a first operating routine (74). The first operating routine may be configured to activate each of the plurality of sensors 26 and communicate sensor data detected by the sensors at a first frequency. As discussed in reference to FIG. 3, the operating routines for the communication circuit 28 and the sensors 26 may be stored and updated in the memory 56 in accordance with the communication control routine 58a and the sensor control routine 58b. While the controller 30 operates the monitoring device 14 via each of the operating routines 58, the system 10 may monitor the energy harvested by the harvesting circuit 20 (76). In some cases, the controller 30 may monitor the energy harvested via a power monitoring routine 58c stored in the memory 56. In other cases, as discussed in reference to FIG. 6B, the performance of the energy harvesting circuit 20 may be monitored over time by the system 10, such that one or more patient-specific or application-specific operating routines may be communicated and loaded into the memory 56 of the controller 30. The application-specific operating routines may include specific reading frequencies for the sensors 26 and communication intervals or times for communicating the sensor data associated with each of the sensors 26. The application-specific operating routines are calculated by software associated with the system 10 (e.g., the controller 30, mobile device 32, computer terminal 36, etc.), such that the sensor data reported by each monitoring device 14 is not only catered to the patient 12 but also limited to the extent that the energy supplied by the harvesting circuit 20 as detected based on the activity and environment of the patient 12 is sufficient to sustain operation of the monitoring device 14.

Referring still to FIG. 6A, as discussed in steps 78-92, the controller 30 may be configured to control the power usage scheme in response to the energy harvested by the harvesting circuit 20. If the power is greater than a first threshold in step 80, the controller 30 may activate the first operating scheme (82). As discussed herein, the control characteristics of each of the operating routines may vary based on the application. As discussed in reference to FIG. 6A, the operating routines are determined based on the energy supplied by the harvesting circuit 20 in reference to various thresholds (e.g., first, second, third, etc.). The first threshold may correspond to a higher level of power supplied by the harvesting circuit 20 than the second threshold. The second threshold is a greater relative power level than the third threshold, and the third threshold is greater than the fourth threshold. When referring to the available power from the harvesting circuit 20, the controller 30 may determine the real-time availability by monitoring the incoming voltage and/or current via one or more circuits in connection with inputs to the controller 30.

If in step 80, the power is less than the first threshold, the method may continue to step 84 to determine if the power is greater than the second threshold. If the power is greater than the second threshold in step 84, the controller may activate the second operating scheme (86). If in step 84, the power is less than the second threshold, the method 70 may continue to step 88 to determine if the power is greater than the third threshold. If the power is greater than the third threshold in step 88, the controller may activate the third operating scheme (90). If in step 88, the power is less than the third threshold, the method 70 may activate the fourth operating scheme (92) and return to step 76 to monitor the power harvested by harvesting circuit 20.

Each of the second, third, and fourth operating schemes may correspond to a diminished operation of the monitoring device 14. To balance the power usage of the monitoring device 14 with the availability of the power harvested by the harvesting circuit 20, the controller 30, or the system 10 more generally, may adjust selectively implement the operating schemes to adjust the operating characteristics of the monitoring device 14. For example, the second operating scheme may maintain or decrease the communication or reporting frequency of the communication circuit 28 for sensor data recorded from each of the sensors 26 relative to a first frequency of the first operating scheme. The third operating scheme may deactivate or limit the operation or reading of one or more of the sensors 26 to further limit the power consumption of the monitoring device 14 relative the first and second operating schemes. In this way, the reading and reporting of information recorded by one or more of the sensor 26 may be prioritized to maintain operation of the monitoring device 14. Finally, the fourth operating scheme may cause the reading and reporting of the sensor data to only be activated periodically with dormant charging intervals for the harvesting circuit 20 executed by the controller 30 between the periodic readings and reports of the sensor data to the system 10. In this way, the system 10 may ensure minimum or essential operation of the monitoring device 14, which may be customized for each patient.

Referring now to FIG. 6B, the method 70 may be configured to forecast and predict an energy availability from the harvesting device 24 over an extended interval (94). The interval may correspond to a periodic interval of patient activity. The length of the interval may be dependent on the specific patient and may correspond to a daily interval, a periodic care or treatment interval (e.g. an exercise frequency, monitoring frequency), or other intervals of time-related to the care or behavior of the patient and the local environment. The length of the interval may be detected based on historical reporting information stored in the memory 56 of the controller 30 or the database 44 of the system 10. Based on the historical or recorded data, the system 10 may identify a forecast for future energy expected to be harvested by the harvesting circuit 20 in connection with the specific patient 12 (96). Once identified, the system 10 may calculate an optimized power consumption or operating scheme for the monitoring device 14. The optimized operating scheme may maximize the recording and reporting or data from the sensors 26 that are most important to the care of the patient 12 based on the availability of power expected to be harvested by harvesting circuit 20. In this way, the system 10 may provide for the communication frequency of the communication circuit 28 and the operation or reading of the sensors 26 to be controlled at calculated frequencies and/or suppressed if necessary. In this way, the system 10 may control the operation of the monitoring device 30 to report the sensor data critical to the patient 12 while maintaining operation of the monitoring device 14 with only the energy from the harvesting devices 24 supplied by the harvesting circuit 20.

As discussed herein, the disclosure provides for a scalable monitoring system that may include one or more of the monitoring devices 14. The system 10 provides for the operation of the monitoring device 14 with energy harvested by the harvesting circuit 20 as disclosed. The disclosure provides for both the reactive management and proactive management of various operating schemes in response to fluctuations in the harvested energy. In this way, the operation of the monitoring device 14 is optimized to control communication and operation of the sensors 26 to operate for weeks or months based on the rate at which power is harvested or the expected energy captured by the harvesting circuit 20. The expected or forecast energy availability or rate of power harvesting is determined based on historical patterns of harvested energy associated with each patient. Accordingly, the system 10 provides for the monitoring device 14 to adjust and automatically sustain operation by automatically adjusting operation and power consumption based on the energy harvesting associated with specific patients. The control of the automatic adjustments may prioritize the detection and reporting of sensor data that is most important for the specific patient.

The system disclosed herein is further summarized in the following paragraphs and is further characterized by combinations of any and all of the various aspects described therein.

According to another aspect of the present disclosure, a sensor apparatus configured to detect at least one physiological parameter of a subject is disclosed. The apparatus includes at least one sensor configured to detect the at least one physiological parameter of the subject as sensor data. A wireless communication circuit is configured to wirelessly communicate the sensor data. The sensor apparatus further includes an energy harvesting circuit with a plurality of energy harvesting devices configured to harvest ambient energy. The energy harvesting devices generate power at a plurality of voltage potential levels from the ambient energy proximate to the subject. At least one conditioning circuit is configured to adjust the plurality of voltage potential levels to a bus voltage supplied to a supply bus. A controller receives operating power via the supply bus and control an activation of the at least one sensor the wireless communication of the sensor data.

According to another aspect of the disclosure, the controller operates exclusively on power harvested by the energy harvesting devices.

According to another aspect of the disclosure, a raw storage unit is in connection with each of the energy harvesting sources that accumulates energy at the voltage potential level output from the connected energy storage device.

According to another aspect of the disclosure, the conditioning circuit periodically converts the energy accumulated in the raw storage units to the bus voltage and conducts the energy into a bulk storage unit or battery storage cell conductively connected to the supply bus.

According to another aspect of the disclosure, the conditioning circuit is in communication with supply bus via a decoupling circuit.

According to another aspect of the disclosure, one of the plurality of energy harvesting devices harvests energy recovered from motion of the apparatus in connection with the subject.

According to another aspect of the disclosure, the controller is further configured to detect a cumulative power harvesting rate of the plurality energy harvesting devices.

According to another aspect of the disclosure, the at least one sensor comprises a plurality of sensors; and the controller is further configured to deactivate one of the sensors in response to the power harvesting rate being less than a threshold rate.

According to another aspect of the disclosure, the controller is further configured to control at least one of an activation of the at least one sensor, a read frequency of the sensor data, a communication frequency of the sensor data, and an awake time of the controller in response to changes in the power harvesting rate.

According to another aspect of the disclosure, the controller is further configured to adjust the power consumption of the sensor apparatus in response to the variations in the supplied energy.

According to another aspect of the disclosure, the controller is further configured to record the supplied energy harvested by the plurality of energy harvesting devices and identify an average power harvested from the plurality of energy harvesting devices over a periodic harvesting period.

According to another aspect of the disclosure, the variations in energy supplied from the plurality of harvesting devices change in response to at least one of a movement of the subject and the environmental conditions proximate to the subject.

According to another aspect of the disclosure, the periodic harvesting period is a calendar day.

According to another aspect of the disclosure, the controller is further configured to control an operating scheme for the at least one of a sensor reading, a communication frequency, and an awake time in response to the average power harvested.

According to another aspect of the disclosure, the at least one sensor comprises at least one of an electrocardiograph (EKG), an electroencephalograph (EEG), an acoustic sensor, an oxygen level sensor, a blood chemistry sensor, a temperature sensors, and a movement detection sensor.

According to another aspect of the disclosure, a system for a sensor apparatus configured to detect at least one physiological parameter of a subject is disclosed. The system includes at least one sensor disposed in the sensor apparatus configured to detect the at least one physiological parameter of the subject as sensor data. A wireless communication circuit is disposed in the sensor apparatus and configured to wirelessly communicate the sensor data to a computerized device. An energy harvesting circuit is also disposed in the sensor apparatus and configured to exclusively supply power the sensor apparatus. The harvesting circuit includes a plurality of energy harvesting devices that harvest energy from at least two different forms of ambient kinetic energy. At least one controller of the system is configured to monitor the energy harvested by the plurality of harvesting devices over a periodic time interval. The at least one controller further predicts a power harvesting rate for the energy harvesting devices over the periodic interval and controls the at least one sensor and the wireless communication circuit with operating power less than the power harvesting rate.

According to another aspect of the disclosure, the at least one controller of the system is further configured to calculate a control scheme comprising a read frequency of the at least one sensor and a communication frequency of the communication circuit that operates the sensor apparatus with the operating power less than the power harvesting rate.

According to another aspect of the disclosure, the at least one sensor comprises a plurality of sensors.

According to another aspect of the disclosure, the at least one controller is further configured to identify a priority of operation for the plurality of sensors based on a monitoring priority for the subject and calculate the control scheme by decreasing an operating frequency of one of the sensors that is a lower priority based on the priority of operation.

According to another aspect of the disclosure, a method for operating a sensor apparatus configured to detect at least one physiological parameter of a subject is disclosed. The method includes monitoring the at least one physiological parameter of the subject as sensor data at a detection frequency with the sensor apparatus and wirelessly communicating the sensor data at a communication frequency. The method further comprises harvesting energy with a plurality of energy harvesting devices. The energy harvesting devices generate power at a plurality of voltage potential levels from ambient energy proximate to the subject. The energy harvested by the energy harvesting devices is the exclusive power supplied to the sensor apparatus. The method further includes adjusting the plurality of voltage potential levels to a bus voltage supplied to a supply bus and supplying the bus voltage to a controller of the sensor apparatus. The controller also controls the detection frequency and the communication frequency.

According to another aspect of the disclosure, the method includes storing and accumulating energy harvested by each of the energy harvesting devices in raw storage units at the voltage potential levels.

According to another aspect of the disclosure, periodically converting the energy accumulated in the raw storage units to the bus voltage.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A sensor apparatus configured to detect at least one physiological parameter of a subject, the apparatus comprising:
at least one sensor configured to detect the at least one physiological parameter of the subject as sensor data;
a wireless communication circuit configured to wirelessly communicate the sensor data;
an energy harvesting circuit comprising:
a plurality of energy harvesting devices configured to harvest ambient energy, wherein the energy harvesting devices generate power at a plurality of voltage potential levels from the ambient energy;
at least one conditioning circuit configured to adjust the plurality of voltage potential levels to a bus voltage supplied to a supply bus; and
a controller that receives operating power via the supply bus, wherein the controller is configured to:
control an activation of the at least one sensor; and
control the wireless communication of the sensor data.

2. The sensor apparatus according to claim 1, wherein the sensor apparatus operates exclusively on power harvested by the energy harvesting devices.

3. The sensor apparatus according to claim 1, further comprising:
a raw storage unit in connection with each of the energy harvesting devices, wherein each of the raw storage units accumulates energy at the voltage potential level output from the connected energy harvesting device.

4. The sensor apparatus according to claim 3, wherein the conditioning circuit periodically converts the energy accumulated in the raw storage units to the bus voltage and conducts the energy into a bulk storage unit or battery storage cell conductively connected to the supply bus.

5. The sensor apparatus according to claim 1, wherein the conditioning circuit is in communication with a supply bus via a decoupling circuit.

6. The sensor apparatus according to claim 1, wherein one of the plurality of energy harvesting devices harvests energy recovered from motion of the apparatus in connection with the subject.

7. The sensor apparatus according to claim 1, wherein the controller is further configured to:
detect a cumulative power harvesting rate of the plurality energy harvesting devices.

8. The sensor apparatus according to claim 7, wherein the at least one sensor comprises a plurality of sensors; and the controller is further configured to deactivate one of the sensors in response to the power harvesting rate being less than a threshold rate.

9. The sensor apparatus according to claim 7, wherein the controller is further configured to:
control at least one of an activation of the at least one sensor, a read frequency of the sensor data, a communication frequency of the sensor data, and an awake time of the controller in response to changes in the power harvesting rate.

10. The sensor apparatus according to claim 1, wherein the controller is further configured to:
monitor variations in a supplied energy harvested by the plurality of energy harvesting devices.

11. The sensor apparatus according to claim 10, wherein the controller is further configured to:
adjust the power consumption of the sensor apparatus in response to the variations in the supplied energy.

12. The sensor apparatus according to claim 10, wherein the controller is further configured to:
record the supplied energy harvested by the plurality of energy harvesting devices; and
identify an average power harvested from the plurality of energy harvesting devices over a periodic harvesting period.

13. The sensor apparatus according to claim 12, wherein the variations in energy supplied from the plurality of harvesting devices change in response to at least one of a movement of the subject and the environmental conditions proximate to the subject.

14. The sensor apparatus according to claim 12, wherein the controller is further configured to:
control an operating scheme for the at least one of a sensor reading, a communication frequency, and an awake time in response to the average power harvested.

15. The sensor apparatus according to claim 1, wherein the at least one sensor comprises at least one of an electrocardiograph (EKG), an electroencephalograph (EEG), an acoustic sensor, an oxygen level sensor, a blood chemistry sensor, a temperature sensors, and a movement detection sensor.

16. A system for a sensor apparatus configured to detect at least one physiological parameter of a subject, the system comprising:
at least one sensor disposed in the sensor apparatus configured to detect the at least one physiological parameter of the subject as sensor data;
a wireless communication circuit disposed in the sensor apparatus configured to wirelessly communicate the sensor data to a computerized device;
an energy harvesting circuit disposed in the sensor apparatus and configured to exclusively supply power to the sensor apparatus, the harvesting circuit comprising a plurality of energy harvesting devices that harvest energy from at least two different forms of ambient kinetic energy; and
at least one controller of the system configured to:
monitor the energy harvested by the plurality of harvesting devices over a periodic time interval;
predict a power harvesting rate for the energy harvesting devices over the periodic interval;
calculate a control scheme comprising a read frequency of the at least one sensor and a communication frequency of the communication circuit that operates the sensor apparatus with the operating power less than the power harvesting rate; and
control the system based on the control scheme.

17. The system according to claim 16, wherein the at least one sensor comprises a plurality of sensors.

18. The system according to claim 16, wherein the at least one controller is further configured to:
identify a priority of operation for the plurality of sensors based on a monitoring priority for the subject; and
calculate the control scheme by decreasing an operating frequency of one of the sensors that is a lower priority based on the priority of operation.

19. A method for operating a sensor apparatus configured to detect at least one physiological parameter of a subject, the method comprising:
detecting the at least one physiological parameter of the subject as sensor data at a detection frequency with the sensor apparatus;
wirelessly communicating the sensor data at a communication frequency;
harvesting energy with a plurality of energy harvesting devices, wherein the energy harvesting devices generate power at a plurality of voltage potential levels from ambient energy, wherein the energy harvested by the energy harvesting devices is the exclusive power supply for the sensor apparatus;
converting the plurality of voltage potential levels to a bus voltage supplied to a supply bus;
supplying the bus voltage to a controller of the sensor apparatus; and
controlling the detection frequency and the communication frequency with the controller.

20. The method according to claim 19, further comprising:
storing and accumulating energy harvested by each of the energy harvesting devices in raw storage units at the voltage potential levels; and
periodically converting the energy accumulated in the raw storage units to the bus voltage.
